(19) United States
(12) Patent Application Publication
(10) Pub. No.: US 2024/0197387 A1
(43) Pub. Date: Jun. 20, 2024

(54) A Directional Radiofrequency (RF) Ablation Needle

(71) Applicant: BOARD OF REGENTS THE UNIVERSITY OF TEXAS SYSTEM, Austin, TX (US)

(72) Inventors: BRIAN T. BOIES, San Antonio, TX (US); MAXIM ECKMANN

(21) Appl. No.: 18/287,013

(22) PCT Filed: Apr. 18, 2022
(86) PCT No.: PCT/US2022/025157

§ 371 Date: Oct. 15, 2023

Related U.S. Application Data
(60) Provisional application No. 63/175,141, filed on Apr. 15, 2021.

(51) Int. Cl. A61B 18/14; A61B 18/00

(52) U.S. Cl. CPC A61B 18/1477; A61B 2018/00077; A61B 2018/00083

(57) ABSTRACT

Certain embodiments are directed to radiofrequency (RF) ablation needle (cannula) design that alters the ablation field CN and enable a directionality to the ablation or RF field, i.e., directional RF needle/cannula. A directional RF ablation cannula described herein can be used to provide specific/directional tissue ablation. Such directionality allows the avoidance of sensitive structures or components of the body by positioning the RF field so that those non-target, sensitive structures or components are not within the ablation field.

### RELATED APPLICATIONS

This application claims priority to U.S. Provisional Patent Application Ser. No. 63/175,141 filed Apr. 15, 2021 which is incorporated herein by reference in its entirety.

### STATEMENT REGARDING FEDERALLY FUNDED RESEARCH

None.

### BACKGROUND

Radiofrequency ablation (RFA) therapy is widely used for tissue ablation. Conventional ablation techniques use radiofrequency (RF) needle electrode(s). The needle electrode(s) can be inserted through bones and tissues into a target area. When deployed, the needle electrode(s) which are electrically connected to a RF generator can transmit RF waves into the surrounding tissue in the target area causing ionic agitation. Ionic agitation occurs around an active electrode resulting in frictional heating in the tissue surrounding the electrode leading to lesions, cell death, and necrosis.

RF ablation probes may be configured in either monopolar or bipolar mode. In monopolar mode, one electrode is located within or on a cannula. To complete the circuit for RF energy, a separate electrode pad or the like is typically placed on the skin of the patient. Bipolar-based devices use multiple electrodes or electrode arrays on a single device. For example, the CONCERTO™ needle electrode device (Boston Scientific Scimed, Inc., Maple Grove, Minn.) uses two electrically independent opposing arrays that are contained within an insulated cannula. RF energy passes between the two arrays and heats the tissue surrounding and in between the arrays.

Typically, the radiofrequency lesions are ellipsoid in nature, and therefore must be placed very carefully to afford the greatest tissue destruction within safe margins. Ellipsoid lesions can damage surrounding healthy tissues, such as nerves and blood vessels. Thus, there is a need for additional radiofrequency ablation needles electrodes that minimize non-target tissue damage.

### SUMMARY

Embodiments described herein provide a solution to minimize or reduce non-specific tissue destruction when using RF ablation needles. Aspects of the invention provide a RF ablation needle design that alters the ablation field and enable a directionality to the ablation or RF field (a directional RF needle). In particular aspects a directional RF ablation needle described herein provides for directional ablation by providing an electrode having proximal to distal insulation, where the insulation has a gap or opening along the long axis of the electrode exposing a portion of the electrode forming a directional RF ablation cannula. The directionality alters the RF field and results in an altered ablation field. A directional RF ablation needle described herein can be used to provide specific/directional tissue ablation. Such directionality allows the avoidance of sensitive structures or components of the body by positioning the RF field so that those non-target, sensitive structures or components are not within the ablation field or minimizes exposure to the ablation field.

Certain embodiments are directed to an electrically insulated radiofrequency ablation needle having an insulating material covering the surface of the needle from a proximal end to a distal end, the insulating material having a gap or opening exposing a portion of the needle/electrode. In certain aspects the gap or opening has a long dimension along the long axis of the cannula and a short dimension perpendicular to the long axis. The gap or opening in the insulating material can be of any geometric shape (triangle, square, circle, oval, rectangle, or other polygonal shape. In certain aspects the gap is a rectangular shaped gap or opening. The long dimension of the gap or opening can be or is at least or at most 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, to 60 mm or more, including all values and ranges there between (i.e., 0.1 to 60 mm, 0.2 to 60 mm, . . . 0.1 to 50 mm, . . . , 50 to 60 mm, etc.). The short dimension can be 0.1, 0.2, 0.3, 0.4, 0.5, 0.6, 0.7, 0.8, 0.9, 1 to 3 mm, including all values and ranges there between (i.e., 0.1 to 3 mm, . . . 0.1 to 1 mm, . . . 0.2 to 3 mm, . . . 1 to 3 mm, etc.). The short dimension can also be expressed as a radial angle of the gap can range from 1, 5, 10, 15, 20, 25, 30, 35, 40, 45, 50, 55, 60, 65, 70, 75, 80, 85, 90, 95, 100, 110, 120, 130, 140, 150, 160, 170, or 180 degrees, including all angles and ranges there between. In certain aspects the ratio of the short dimension to long dimension can be 1:10, 1:9, 1:8, 1:7, 1:6, 1:5, 1:4, 1:3, 1:2, or 1:1, including all ratios and ranges there between (i.e., 1 to 180 degrees, . . . 5 to 180 degrees, . . . 170 to 180 degrees, etc.). In certain aspects, the gap or opening can be square, rectangular, circular, triangular, or polygonal. In particular aspects the gap or opening is rectangular with the long axis of the rectangle along the long axis of the needle. In certain aspects the gap or opening terminates at the distal end of the needle. In other aspects the gap or opening terminates before the distal end of the needle. When the opening terminates before the distal end there can be 0.1, 0.2, 0.3, 0.4, 0.5, 0.6, 0.7, 0.8, 0.9, 1 to 3 mm, including all values and ranges there between, of insulating material between the end of the gap or opening and the distal end of the needle. The insulation layer can have an average thickness 0.01, 0.1, 1, 1.5, to 2 mm, including all values and ranges there between. The gap can also be characterized as the radial angle of non-insulated surface relative to the circumference of the needle. The radial angle of the gap can range from 1, 5, 10, 15, 20, 25, 30, 35, 40, 45, 50, 55, 60, 65, 70, 75, 80, 85, 90, 95, 100, 110, 120, 130, 140, 150, 160, 170, or 180 degrees, including all angles and ranges there between. In certain aspects the radial angle is 10 to 20 degrees and in some embodiments 15±2 degrees. In certain aspects the directional RF ablation needle has a length 5, 6, 7, 8, 9, 10, 11, 12, 13, 14, to 15 cm.

In certain aspects, the proximal end of the directional RF ablation needle can be configured to connect to a radiofrequency generator.

The term “radiofrequency ablation” refers to a therapeutic method in which a target tissue within a subject body, is cauterized and necrotized by radiofrequency heating without being excised.

The term “radiofrequency ablation lesion” refers to the lesion caused by radiofrequency ablation method.

The term “ablation” as used herein means thermal damage to the tissue causing tissue or cell necrosis.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration comparing traditional radiofrequency ablation needle and directional radiofrequency ablation needle.

FIG. 2. Illustration of tissue ablation field comparison between traditional radiofrequency ablation needle and directional radiofrequency ablation needle.

FIG. 3. Illustration describing the narrowing of the α angle associated with directional radiofrequency needles.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

The present disclosure may be further understood with reference to the appended drawings and the following description, wherein like elements are referred to with the same reference numerals. The present disclosure relates to devices and methods for ablating tissue. It should be noted that the terms proximal and distal, as used herein, are intended to refer to a direction toward (proximal) and away from (distal) a user of the device (e.g., physician).

The present disclosure relates to an ablation device which includes a needle having a proximal end and a distal end. In certain aspects the proximal end remains outside a body during use and is accessible to a user. In certain aspects the distal end is a tissue penetrating distal end and configured as at least a first ablation electrode. In certain aspects the directional RF needle includes an external insulation layer. The external insulation layer is an electrically insulative layer. The needle can be formed of an electrically conductive material with the insulation layer circumferentially therearound. The insulation layer extending to the distal end of the needle. The directionality of the RF field is imparted by a gap or opening in the insulation layer that provides for a directional RF field. In certain embodiments the directional RF needle is positioned inside of a delivery cannula, with the ablation needle being capable of being extended beyond the tip of the delivery cannula during operation. In other aspects, the ablation needle can be retracted into the delivery cannula.

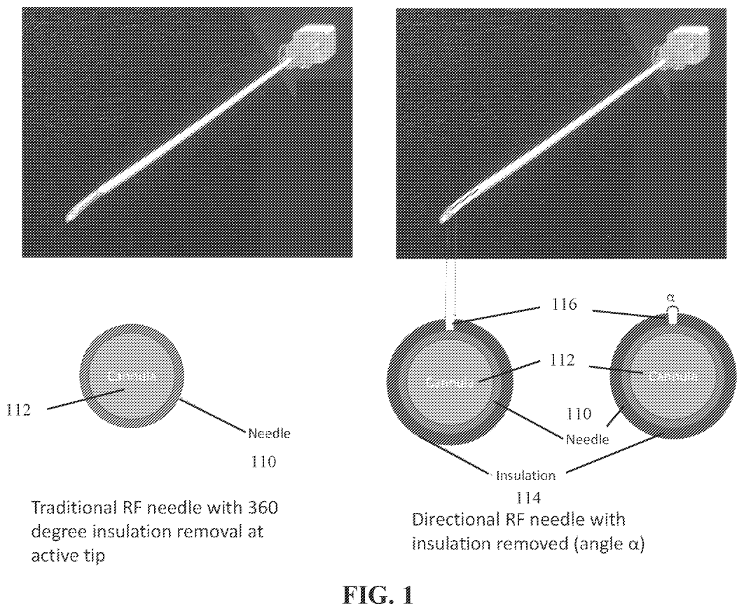

An example of a directional RF needle is provided in FIG. 1. FIG. 1 illustrates a traditional RF needle and a directional RF needle. A cross section via of the RF needles is provided. The cross section illustrates the relationship between needle 10, cannula/lumen 12, and insulation 14 in the case of the directional RF needle. In certain aspects the directional RF needle includes insulation layer 14. The external insulation layer 14 is an electrically insulative layer. Needle 10 is formed of an electrically conductive material with the insulation layer 14 circumferentially there around. The insulation layer 14 extending to the distal end of needle 10. The directionality of the RF field is imparted by gap or opening 16 in the insulation layer 14, the gap provides for a directional RF field.

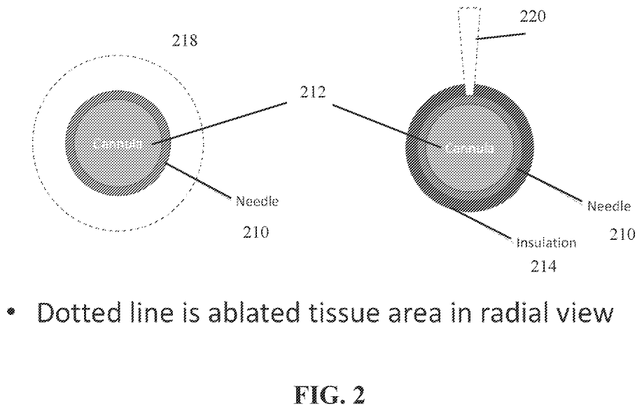

FIG. 2 illustrates the ablation fields produced by a traditional RF needle and a directional RF needle. The non-insulated tip of the traditional needle 210 generating ablation field 218 that extends 360° relative to the long axis of the uninsulated portion of needle 210. The insulated tip of the directional RF needle can produce a narrowed ablation field 220 as a result of the gap or opening 216 in the insulation layer 214.

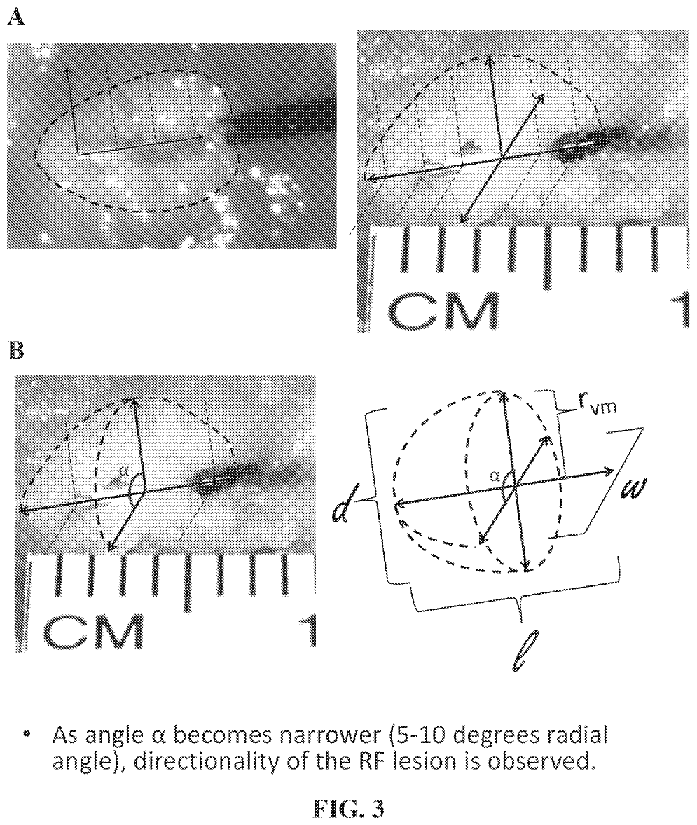

FIG. 3 provides an illustration of the narrowing of ablation field in terms the angle α. Angle α is the amount of linear radial insulation defect measured out of a full 360 degree circle, with the axial center of the cannula being the reference point for rotation. In this example α can be a radial angle of 5 to 10 degrees. The radial angle can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, to 180 degrees, including all values and ranges there between.

In certain embodiments, an ablation device can further includes a second ablation needle (second electrode) separated the first ablation needle (first electrode), the first and second needles/electrodes being coupled to opposite poles of a power source to function as a bi-polar ablation system.

In certain aspects the second ablation needle is a directional RF ablation needle and includes an external insulation layer. The needle can be formed of an electrically conductive material with the insulation layer circumferentially there around. The insulation layer extending to the distal end of the cannula. The directionality of the RF field is imparted by a gap or opening in the insulation layer that provides for a directional RF field. In certain embodiments the needle is positioned inside of a delivery cannula, with the ablation needle being capable of being extended beyond the tip of the delivery cannula during operation. In other aspects, the ablation needle can be retracted into the delivery cannula.

A directional RF needle can comprise an electrically conductive needle portion. The needle portion can be formed of a biocompatible and electrically conductive material such as stainless steel, nitinol, Inconel, platinum and other biocompatible electrically conductive materials. The insulation layer can have a thickness of 0.0001 to 1 mm, including all values and ranges there between. In certain aspects, the insulation layer can be a layer or coating of polyvinyl carboxylate, polyvinyl acetal, polystyrene, polycarbonate, polyarylate, polyester, polyamide, polyimide, polyurethane, polysiloxane, polysulfone, polymethyl methacrylate, polymethyl acrylate, cellulose, polyethylene, polypropylene, a copolymer of these, rubber, and a thermoplastic elastomer or any other insulating polymer. In certain aspects, the insulating layer is polyethylene terephthalate (PET) or polytetrafluoroethylene.

The ablation needle(s) receives electrical energy supplied to a proximal end of the ablation needle and produces a directional RF field for ablating target tissue adjacent to the exposed portion of the ablation needle. In certain aspects, the ablation needle is coupled with a delivery element/cannula. In certain aspects the insulation layer has a length selected so that, when the ablation needle is in use a portion of the insulation layer remains within the delivery element/cannula to ensure that energy is delivered to tissue only via the exposed portion. In certain aspects, mechanisms are employed to advance and retract the ablation needle relative to the delivery element/cannula. A power source and controller may be used to supply the RF ablation energy to the ablation needle. For example, a Boston Scientific RF3000™ or similar device may be used as the power source for any of the embodiments described herein. In certain aspects, the lesion can be partial or hemi oblate ellipsoid in shape.

During use, the distal portion of the ablation needle can be used to penetrate and be positioned in a target tissue. In certain aspects, any known visualization system can be used to guide the position of the ablation needle. Once in position power may be supplied to the ablation needle as needed to produce the appropriate lesion. Once the target has been ablated, the power supply is terminated and the ablation needle is withdrawn. In certain aspects, the operator would use an indicator on the hub of the needle, the indicator indicating the direction of the ablation zone. The user would align this zone toward the indicated target with deliberate needle rotation using the indicator mark as a guide in conjunction with anatomic knowledge and relevant imaging. After confirmation that the needle rotation is in proper position, the user would proceed with directional lesioning.

A directional RF ablation needle as described herein can be selected or designed based on application parameters such as the size of the lesion that is desired, an amount of power that can safely be delivered, and constraints of the anatomy. In certain aspects, a first and/or second ablation needle can be 1 cm in length and separated from one another by a gap of 1 cm. Each ablation needle having an outer diameter of approximately 1 mm. The insulation layer can be about 0.125 mm. Any known power source and controller may be used to supply the RF ablation energy to the ablation needle.

1. A directional radiofrequency (RF) ablation needle, comprising:
a conductive body having a proximal end and distal end, the conductive body have an outer diameter of 0.1 to 20 mm and a length of 2 to 30 cm;
an insulation layer on an outer surface of the conductive body extending from the proximal end to the distal end of the conductive body; and
an exposed portion of the conductive body formed by a gap or opening in the insulation layer, the exposed portion having a circumferential angle of exposed conductive body of 1 to 180 degrees,
wherein the directional RF ablation needle is configured to produce an asymmetric electromagnetic field.

2. The ablation needle of claim 1, wherein opening the opening is 0.1 mm to 50 mm along a long axis of the conductive body.

3. The ablation needle of claim 1, wherein
opening the opening is 0.1 mm to 10 mm along a long axis of the conductive body.

4. The ablation needle of claim 1, wherein the conductive body is 10 mm to 60 mm in length.

5. The ablation needle of claim 1, wherein the insulation layer has an average thickness 0.01 mm to 1 mm.

6. The ablation needle of claim 1, wherein the exposed portion of the conductive body has a circumferential angle of 1 to 90 degrees.

7. The ablation needle of claim 1, wherein the exposed portion of the conductive body has a circumferential angle of 1 to 45 degrees.

8. The ablation needle of claim 1, wherein the exposed portion of the conductive body has a circumferential angle of 1 to 15 degrees.

9. The ablation needle of claim 1, further comprising a radiofrequency generator connector at the proximal end of the conductive body.

10. The ablation needle of claim 1, wherein the insulation layer is a layer or coating of polyvinyl carboxylate, polyvinyl acetal, polystyrene, polycarbonate, polyarylate, polyester, polyamide, polyimide, polyurethane, polysiloxane, polysulfone, polymethyl methacrylate, polymethyl acrylate, cellulose, polyethylene, polypropylene, a copolymer of these, rubber, and a thermoplastic elastomer.

11. The ablation needle of claim 1, wherein the insulation layer is a layer or coating of polyethylene terephthalate (PET) or polytetrafluoroethylene.

12. The ablation needle of claim 1, further comprising a thermocouple.

13. A radiofrequency ablation system comprising the ablation needle of claim 1, a radiofrequency generator, and an ablation switching controller.
